(19) United States
(12) Patent Application Publication
(10) Pub. No.: US 2012/0178720 A1
(43) Pub. Date: Jul. 12, 2012

(54) Bisphosphonates as Inhibitors of Acid Sphingomyelinase

(76) Inventors: Christoph Arenz, Berlin (DE); Anke Gundula Roth, Berlin (DE); Stefan Uhlig, Aachen (DE); Daniela Drescher, Berlin (DE)

(21) Appl. No.: 13/391,328

(22) PCT Filed: Aug. 19, 2010
(86) PCT No.: PCT/EP10/62134

§ 371 Date: Mar. 13, 2012

(30) Foreign Application Priority Data
Aug. 26, 2009 (EP) 09168712.9

(51) Int. Cl. A61K 31/663; A61P 11/00; A61P 9/10; C12N 9/99

(52) U.S. Cl. 514/108; 435/184

(57) ABSTRACT

The present invention refers to bisphosphonate and phosphonate/phosphate compounds of Formulae I and its use as inhibitors of aSMase enzyme activity.

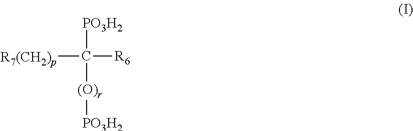

The acid sphingomyelinase (aSMase) is a soluble lysosomal sphingolipid hydrolase, which constitutively degrades sphingomyelin from internalized membrane fragments (T. Kolter, K. Sandhoff, Angew. Chem. 1999, 111, 1632; Angew Chem Int Ed 1999, 38, 1532). Upon stimulation, a portion of this enzyme can be found at the outer side of the plasma membrane (S. Marathe, S. L. Schissel, M. J. Yellin, N. Beatini, R. Mintzer, K. J. Williams, I. Tabas, J. Biol. Chem. 1998, 273, 4081). This membrane-associated enzyme shows biochemical activity in serum and urine. Its activity is elevated in several diseases. The secretory form of aSMase is believed to play an important role in signal transduction, since it alters the composition of the plasma membrane within putative sphingolipid- and cholesterol-rich membrane micro-domains. These so-called ‘lipid rafts’ have been suggested to act as ‘signalling platforms’ (K. Simons, E. Ikonen, Nature 1997, 387, 569) and there is significant evidence, that the cleavage of sphingomyelin to ceramide is able to dramatically alter the biophysical properties of the putative rafts (Megha, E. London, J Biol Chem 2004, 279, 9997). Alternatively, it is proposed that ceramide acts by binding to intra-cellular proteins like cathepsin D, ceramide activated protein phosphatases (CAPP), phospholipase A2, protein kinase c isoforms, kinase suppressor of Ras (KSR) and c-Raf-1 (H. Grassmé, K A Becker, Y Zhang, E. Gulbins Ceramide in bacterial infections and cystic fibrosis Biol. Chem. 2008 389, 1371-9).

The aSMase is emerging as an important drug target in a variety of diseases. Amongst others, it has been shown that inhibition of aSMase prevents bacterial infections in a rat model of cystic fibrosis and formation of acute lung injury (ALI) elicited by endotoxin, acid instillation or platelet-activating factor (PAF). Moreover, the aSMase is essential for infection of non-phagocytotic cells with Neisseria gonorrhoea and formation of pulmonary emphysema. Pharmacological or genetic inhibition of aSMase prevents apoptosis and degeneration of liver cells in a mouse model for Wilson's disease. In addition, there are several reports that aSMase significantly contributes to the formation of atherosclerotic plaques.

However, this promising progress in aSMase-research, based on sophisticated animal models and cultured patient's cells, is thwarted by the lack of potent and selective inhibitors of this enzyme. Phosphatidylinositol-3,5-bisphosphate (PtdIns-3,5P₂), the most potent inhibitor (M. Kolzer, C. Arenz, K. Ferlinz, N. Werth, H. Schulze, R. Klingenstein, K. Sandhoff, Biol Chem 2003, 384, 1293.), is not suited for cell culture studies or straight forward in vivo application, because of its 5-fold negative charge and its two long fatty acid chains causing it to stack in cellular membranes. Last but not least, this inhibitor is labile towards phospholipases A₁, A₂, C and D and phosphoinositide phosphatases.

It is an object of the present invention to provide novel potent inhibitors of acid sphingomyelinase.

It has surprisingly been found that compounds of Formula I are efficient inhibitors of aSMase enzyme activity.

Compounds of Formula I comprise geminal bisphosphonates and mixed phosphonate/phosphate compounds described by:

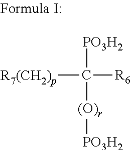

wherein
p is an integer from 4 to 12;
r is 0 or 1;
R₆=H, OH, NH₂ or N(CH₃)₂; and
R₇=CH₃, NH₂ or N(CH₃)₂; preferably R₇ is CH₃.

Preferably R₆ is H, OH or NH₂, more preferably R₆ is OH or NH₂. The integer p can be from 4 to 12, preferably from 5 to 10.

Preferred compounds of Formula I are the compounds:
H₃C(CH₂)₄C(PO₃H₂)₂H
H₃C(CH₂)₅C(PO₃H₂)₂H
H₃C(CH₂)₆C(PO₃H₂)₂H
H₃C(CH₂)₇C(PO₃H₂)₂H
H₃C(CH₂)₈C(PO₃H₂)₂H
H₃C(CH₂)₉C(PO₃H₂)₂H
H₃C(CH₂)₁₀C(PO₃H₂)₂H
H₃C(CH₂)₁₁C(PO₃H₂)₂H
H₃C(CH₂)₄C(PO₃H₂)₂OH
H₃C(CH₂)₅C(PO₃H₂)₂OH
H₃C(CH₂)₆C(PO₃H₂)₂OH
H₃C(CH₂)₇C(PO₃H₂)₂OH
H₃C(CH₂)₈C(PO₃H₂)₂OH
H₃C(CH₂)₉C(PO₃H₂)₂OH
H₃C(CH₂)₁₀C(PO₃H₂)₂OH
H₃C(CH₂)₁₁C(PO₃H₂)₂OH
H₃C(CH₂)₄C(PO₃H₂)₂NH₂
H₃C(CH₂)₅C(PO₃H₂)₂NH₂
H₃C(CH₂)₆C(PO₃H₂)₂NH₂
H₃C(CH₂)₇C(PO₃H₂)₂NH₂
H₃C(CH₂)₈C(PO₃H₂)₂NH₂
H₃C(CH₂)₉C(PO₃H₂)₂NH₂
H₃C(CH₂)₁₀C(PO₃H₂)₂NH₂
H₃C(CH₂)₁₁C(PO₃H₂)₂NH₂
H₃C(CH₂)₄C(PO₃H₂)₂N(CH₃)₂
H₃C(CH₂)₅C(PO₃H₂)₂N(CH₃)₂
H₃C(CH₂)₆C(PO₃H₂)₂N(CH₃)₂
H₃C(CH₂)₇C(PO₃H₂)₂N(CH₃)₂
H₃C(CH₂)₈C(PO₃H₂)₂N(CH₃)₂
H₃C(CH₂)₉C(PO₃H₂)₂N(CH₃)₂
H₃C(CH₂)₁₀C(PO₃H₂)₂N(CH₃)₂
H₃C(CH₂)₁₁C(PO₃H₂)₂N(CH₃)₂
H₃C(CH₂)₄C(PO₃H₂)(PO₄H₂)H
H₃C(CH₂)₅C(PO₃H₂) (PO₄H₂)H
H₃C(CH₂)₆C(PO₃H₂)(PO₄H₂)H
H₃C(CH₂)₇C(PO₃H₂)(PO₄H₂)H
H₃C(CH₂)₈C(PO₃H₂)(PO₄H₂)H
H₃C(CH₂)₉C(PO₃H₂)(PO₄H₂)H
H₃C(CH₂)₁₀C(PO₃H₂)(PO₄H₂)H
H₃C(CH₂)₁₁C(PO₃H₂)(PO₄H₂)H
H₃C(CH₂)₄C(PO₃H₂)(PO₄H₂)OH
H₃C(CH₂)₅C(PO₃H₂)(PO₄H₂)OH
H₃C(CH₂)₆C(PO₃H₂)(PO₄H₂)OH
H₃C(CH₂)₇C(PO₃H₂)(PO₄H₂)OH
H₃C(CH₂)₈C(PO₃H₂)(PO₄H₂)OH
H₃C(CH₂)₉C(PO₃H₂)(PO₄H₂)OH
H₃C(CH₂)₁₀C(PO₃H₂)(PO₄H₂)OH
H₃C(CH₂)₁₁C(PO₃H₂)(PO₄H₂)OH
H₃C(CH₂)₄C(PO₃H₂)(PO₄H₂)NH₂
H₃C(CH₂)₅C(PO₃H₂)(PO₄H₂)NH₂
H₃C(CH₂)₆C(PO₃H₂)(PO₄H₂)NH₂
H₃C(CH₂)₇C(PO₃H₂)(PO₄H₂)NH₂
H₃C(CH₂)₈C(PO₃H₂)(PO₄H₂)NH₂
H₃C(CH₂)₉C(PO₃H₂)(PO₄H₂)NH₂
H₃C(CH₂)₁₀C(PO₃H₂)(PO₄H₂)NH₂
H₃C(CH₂)₁₁C(PO₃H₂)(PO₄H₂)NH₂
H₃C(CH₂)₄C(PO₃H₂)(PO₄H₂)N(CH₃)₂
H₃C(CH₂)₅C(PO₃H₂)(PO₄H₂)N(CH₃)₂
H₃C(CH₂)₆C(PO₃H₂)(PO₄H₂)N(CH₃)₂
H₃C(CH₂)₇C(PO₃H₂)(PO₄H₂)N(CH₃)₂
H₃C(CH₂)₈C(PO₃H₂)(PO₄H₂)N(CH₃)₂
H₃C(CH₂)₉C(PO₃H₂)(PO₄H₂)N(CH₃)₂
H₃C(CH₂)₁₀C(PO₃H₂)(PO₄H₂)N(CH₃)₂; and/or
H₃C(CH₂)₁₁C(PO₃H₂)(PO₄H₂)N(CH₃)₂;
as well as all compounds described in Table 2.

Other preferred compounds of Formula I are:
NH₂(CH₂)₄C(PO₃H₂)₂OH
NH₂(CH₂)₅C(PO₃H₂)₂OH
NH₂(CH₂)₆C(PO₃H₂)₂OH
NH₂(CH₂)₇C(PO₃H₂)₂OH
NH₂(CH₂)₈C(PO₃H₂)₂OH
NH₂(CH₂)₉C(PO₃H₂)₂OH
NH₂(CH₂)₁₀C(PO₃H₂)₂OH
NH₂(CH₂)₁₁C(PO₃H₂)₂OH
N(CH₃)₂(CH₂)₄C(PO₃H₂)₂OH
N(CF₃)₂(CH₂)₅C(PO₃H₂)₂OH
N(CH₃)₂(CH₂)₆C(PO₃H₂)₂OH
N(CH₃)₂(CH₂)₇C(PO₃H₂)₂OH
N(CH₃)₂(CH₂)₈C(PO₃H₂)₂OH
N(CH₃)₂(CH₂)₉C(PO₃H₂)₂OH
N(CH₃)₂(CH₂)₁₀C(PO₃H₂)₂OH; and/or
N(CH₃)₂(CH₂)₁₁C(PO₃H₂)₂OH, wherein compounds NH₂(CH₂)₄C(PO₃H₂)₂OH and/or
NH₂(CH₂)₁₀C(PO₃H₂)₂OH are particularly preferred.

Further preferred compounds of Formula I are:
H₃C(CH₂)₄C(PO₃H₂)₂H
H₃C(CH₂)₅C(PO₃H₂)₂H
H₃C(CH₂)₆C(PO₃H₂)₂H
H₃C(CH₂)₇C(PO₃H₂)₂H
H₃C(CH₂)₈C(PO₃H₂)₂H
H₃C(CH₂)₉C(PO₃H₂)₂H
H₃C(CH₂)₁₀C(PO₃H₂)₂H
H₃C(CH₂)₁₁C(PO₃H₂)₂H
H₃C(CH₂)₆C(PO₃H₂)₂OH
H₃C(CH₂)₁₁C(PO₃H₂)₂OH
H₃C(CH₂)₆C(PO₃H₂)₂NH₂
H₃C(CH₂)₇C(PO₃H₂)₂NH₂
H₃C(CH₂)₈C(PO₃H₂)₂NH₂
H₃C(CH₂)₉C(PO₃H₂)₂NH₂
H₃C(CH₂)₁₀C(PO₃H₂)₂NH₂
H₃C(CH₂)₁₁C(PO₃H₂)₂NH₂
H₃C(CH₂)₄C(PO₃H₂)₂N(CH₃)₂
H₃C(CH₂)₅C(PO₃H₂)₂N(CH₃)₂
H₃C(CH₂)₆C(PO₃H₂)₂N(CH₃)₂
H₃C(CH₂)₇C(PO₃H₂)₂N(CH₃)₂
H₃C(CH₂)₈C(PO₃H₂)₂N(CH₃)₂
H₃C(CH₂)₉C(PO₃H₂)₂N(CH₃)₂
H₃C(CH₂)₁₀C(PO₃H₂)₂N(CH₃)₂
H₃C(CH₂)₁₁C(PO₃H₂)₂N(CH₃)₂
H₃C(CH₂)₄C(PO₃H₂)(PO₄H₂)H
H₃C(CH₂)₅C(PO₃H₂)(PO₄H₂)H
H₃C(CH₂)₆C(PO₃H₂)(PO₄H₂)H
H₃C(CH₂)₇C(PO₃H₂)(PO₄H₂)H
H₃C(CH₂)₈C(PO₃H₂)(PO₄H₂)H
H₃C(CH₂)₉C(PO₃H₂)(PO₄H₂)H
H₃C(CH₂)₁₀C(PO₃H₂)(PO₄H₂)H
H₃C(CH₂)₁₁C(PO₃H₂)(PO₄H₂)H
H₃C(CH₂)₄C(PO₃H₂)(PO₄H₂)OH
H₃C(CH₂)₅C(PO₃H₂)(PO₄H₂)OH
H₃C(CH₂)₆C(PO₃H₂)(PO₄H₂)OH
H₃C(CH₂)₇C(PO₃H₂)(PO₄H₂)OH
H₃C(CH₂)₈C(PO₃H₂)(PO₄H₂)OH
H₃C(CH₂)₉C(PO₃H₂)(PO₄H₂)OH
H₃C(CH₂)₁₀C(PO₃H₂)(PO₄H₂)OH
H₃C(CH₂)₁₁C(PO₃H₂)(PO₄H₂)OH
H₃C(CH₂)₄C(PO₃H₂)(PO₄H₂) NH₂
H₃C(CH₂)₅C(PO₃H₂)(PO₄H₂)NH₂
H₃C(CH₂)₆C(PO₃H₂)(PO₄H₂)NH₂
H₃C(CH₂)₇C(PO₃H₂)(PO₄H₂)NH₂
H₃C(CH₂)₈C(PO₃H₂)(PO₄H₂)NH₂
H₃C(CH₂)₉¹C(PO₃H₂)(PO₄H₂)NH₂
H₃C(CH₂)₁₀C(PO₃H₂)(PO₄H₂)NH₂
H₃C(CH₂)₁₁C(PO₃H₂)(PO₄H₂)NH₂
H₃C(CH₂)₄C(PO₃H₂)(PO₄H₂)N(CH₃)₂
H₃C(CH₂)₅C(PO₃H₂)(PO₄H₂)N(CH₃)₂
H₃C(CH₂)₆C(PO₃H₂)(PO₄H₂)N(CH₃)₂
H₃C(CH₂)₇C(PO₃H₂)(PO₄H₂)N(CH₃)₂
H₃C(CH₂)₈C(PO₃H₂)(PO₄H₂)N(CH₃)₂
H₃C(CH₂)₉C(PO₃H₂)(PO₄H₂)N(CH₃)₂
H₃C(CH₂)₁₀C(PO₃H₂)(PO₄H₂)N(CH₃)₂; and/or
H₃C(CH₂)₁₁C(PO₃H₂)(PO₄H₂)N(CH₃)₂.

Other preferred compounds of Formula I are:
H₃C(CH₂)₄C(PO₃H₂)₂H
H₃C(CH₂)₆C(PO₃H₂)₂H
H₃C(CH₂)₇C(PO₃H₂)₂H
H₃C(CH₂)₈C(PO₃H₂)₂H H₃C(CH₂)₉C(PO₃H₂)₂H
H₃C(CH₂)₁₀C(PO₃H₂)₂H
H₃C(CH₂)₁₁C(PO₃H₂)₂H
H₃C(CH₂)₉C(PO₃H₂)₂NH₂
H₃C(CH₂)₁₁C(PO₃H₂)₂NH₂
H₃C(CH₂)₆C(PO₃H₂)₂N(CH₃)₂
H₃C(CH₂)₇C(PO₃H₂)₂N(CH₃)₂
H₃C(CH₂)₈C(PO₃H₂)₂N(CH₃)₂
H₃C(CH₂)₉C(PO₃H₂)₂N(CH₃)₂
H₃C(CH₂)₁₀C(PO₃H₂)₂N(CH₃)₂;
H₃C(CH₂)₁₁C(PO₃H₂)₂N(CH₃)₂;
H₃C(CH₂)₄C(PO₃H₂)(PO₄H₂)H
H₃C(CH₂)₅C(PO₃H₂)(PO₄H₂)H
H₃C(CH₂)₆C(PO₃H₂)(PO₄H₂)H
H₃C(CH₂)₇C(PO₃H₂)(PO₄H₂)H
H₃C(CH₂)₈C(PO₃H₂)(PO₄H₂)H
H₃C(CH₂)₉C(PO₃H₂)(PO₄H₂)H
H₃C(CH₂)₁₀C(PO₃H₂)(PO₄H₂)H
H₃C(CH₂)₁₁C(PO₃H₂)(PO₄H₂)H
H₃C(CH₂)₄C(PO₃H₂)(PO₄H₂)OH
H₃C(CH₂)₅C(PO₃H₂)(PO₄H₂)OH
H₃C(CH₂)₆C(PO₃H₂)(PO₄H₂)OH
H₃C(CH₂)₇C(PO₃H₂)(PO₄H₂)OH
H₃C(CH₂)₈C(PO₃H₂)(PO₄H₂)OH
H₃C(CH₂)₉C(PO₃H₂)(PO₄H₂)OH
H₃C(CH₂)₁₀C(PO₃H₂)(PO₄H₂)OH
H₃C(CH₂)₁₁C(PO₃H₂)(PO₄H₂)OH
H₃C(CH₂)₄C(PO₃H₂)(PO₄H₂)NH₂
H₃C(CH₂)₅C(PO₃H₂)(PO₄H₂)NH₂
H₃C(CH₂)₆C(PO₃H₂)(PO₄H₂)NH₂
H₃C(CH₂)₇C(PO₃H₂)(PO₄H₂)NH₂
H₃C(CH₂)₈C(PO₃H₂)(PO₄H₂)NH₂
H₃C(CH₂)₉C(PO₃H₂)(PO₄H₂)NH₂
H₃C(CH₂)₁₀C(PO₃H₂)(PO₄H₂)NH₂
H₃C(CH₂)₁₁C(PO₃H₂)(PO₄H₂)NH₂
H₃C(CH₂)₄C(PO₃H₂)(PO₄H₂)N(CH₃)₂
H₃C(CH₂)₅C(PO₃H₂)(PO₄H₂)N(CH₃)₂
H₃C(CH₂)₆C(PO₃H₂)(PO₄H₂)N(CH₃)₂
H₃C(CH₂)₇C(PO₃H₂)(PO₄H₂)N(CH₃)₂
H₃C(CH₂)₈C(PO₃H₂)(PO₄H₂)N(CH₃)₂
H₃C(CH₂)₉C(PO₃H₂)(PO₄H₂)N(CH₃)₂
H₃C(CH₂)₁₀C(PO₃H₂)(PO₄H₂)N(CH₃)₂; and/or
H₃C(CH₂)₁₁C(PO₃H₂)(PO₄H₂)N(CH₃)₂.

Since compounds of Formula I and preferred compounds thereof inhibit enzyme activity of aSMase in vitro and in vivo, these compounds can be used as inhibitors of aSMase. In particular a compound of Formula I or preferred compounds thereof can be used for inhibition of acid sphingomyelinase enzyme activity in vitro. The term “in vitro” refers to any use or method not practised on the human or animal body. Such a use encompasses the use of compounds of Formula I or II in a cellular or cell-free assay for aSMase activity.

Compounds of Formula I or preferred compounds thereof may be used as a medicament, in particular as a medicament for treatment, diagnosis and/or prophylaxis of a disease associated with altered, elevated or unwanted aSMase enzyme activity.

It has already been documented that aSMase enzyme activity plays a crucial role in a number of diseases. Diseases which have already been associated with aSMase activity comprise e.g.:
infectious diseases like bacterial infections, e.g. with infection with Neisseria gonnorhoeae (H. Grassme, E. Gulbins, B. Brenner, K. Ferlinz, K. Sandhoff, K. Harzer, F. Lang, T. F. Meyer, Cell 1997, 91, 605);
lung diseases like acute lung injury (ALI) or acute respiratory distress syndrome (ARDS), lung oedema, (R. Goggel, S. Winoto-Morbach, G. Vielhaber, Y. Imai, K. Lindner, L. Brade, H. Brade, S. Ehlers, A. S. Slutsky, S. Schutze, E. Gulbins, S. Uhlig, Nat Med 2004, 10, 155);
pulmonary emphysema (I. Petrache, V. Natarajan, L. Zhen, T. R. Medler, A. T. Richter, C. Cho, W. C. Hubbard, E. V. Berdyshev, R. M. Tuder, Nat Med 2005, 11, 491);
infections, e.g. bacterial infections, during cystic fibrosis (V. Teichgraber, M. Ulrich, N. Endlich, J. Riethmuller, B. Wilker, C. C. De Oliveira-Munding, A. M. van Heeckeren, M. L. Barr, G. von Kurthy, K. W. Schmid, M. Weller, B. Tummler, F. Lang, H. Grassme, G. Doring, E. Gulbins, Nat Med 2008, 14, 382);
Morbus Wilson (P. A. Lang, M. Schenck, J. P. Nicolay, J. U. Becker, D. S. Kempe, A. Lupescu, S. Koka, K. Eisele, B. A. Klarl, H. Rubben, K. W. Schmid, K. Mann, S. Hildenbrand, H. Hefter, S. M. Huber, T. Wieder, A. Erhardt, D. Haussinger, E. Gulbins, F. Lang, Nat Med 2007, 13, 164);
atherosclerosis, coronary heart disease, cardiovascular diseases (C. M. Devlin, A. R. Leventhal, G. Kuriakose, E. H. Schuchman, K. J. Williams, I. Tabas, Arterioscler Thromb Vase Biol 2008, 28, 1723);
diabetes type II (Gorska, M., Baranczuk, E., and Dobrzyn, A. (2003) Norm. Metab. Res. 35, 506-507; Straczkowski, M., Kowalska, I., Baranowski, M., Nikolajuk, A., Otziomek, E., Zabielski, P., Adamska, A., Blachnio, A., Gorski, J., and Gorska, M. (2007) Increased skeletal muscle ceramide level in men at risk of developing type 2 diabetes. Diabetologia 50, 2366-2373);
major depression (Kornhuber, J., Medlin, A., Bleich, S., Jendrossek, V., Henkel, A. W., Wiltfang, J., and Gulbins, E. (2005) High activity of acid sphingomyelinase in major depression. J. Neural. Transm. 112, 1583-1590);
Alzheimer disease (Han, X. (2005) Lipid alterations in the earliest clinically recognizable stage of Alzheimer's disease: implication of the role of lipids in the pathogenesis of Alzheimer's disease. Curr. Alzheimer Res. 2, 65-77);
Niemann-Pick disease (E. Schuchman, R. J. Desnick, in The Metabolic Basis of Inherited Disease (Eds.: C. Scriver, W. Sly, D. Valle), McGraw Hill, New York, 2001, pp. 3589; J. Q. Fan, Trends Pharmacol Sci 2003; 24, 355; E. Schuchman, R. J. Desnick, 2005, p. WO 2005/051331.)
Cancer (T. Kirkegaard, A. G. Roth, N. H. T. Petersen, A. K. Mahalka, O. D. Olsen, I. Moilanen, A. Zylicz, J. Knudsen, K. Sandhoff, C. Arenz, P. K. J. Kinnunen, J. Nylandsted, M. Jäättelä (2005) Hsp70 stabilizes lysosomes and reverts Niemann-Pick disease-associated lysosomal pathology, Nature 463, 549-554.)

Niemann-Pick Disease (NPD) Type A and B is one of several known lysosomal storage diseases. It is a rare, recessively inherited disease caused by mutations in the gene coding for the acid sphingomyelinase (aSMase) leading to a partial loss of functional enzyme in the lysosomes. As a consequence, sphingomyelin, a major constituent of eukaryotic plasma membranes and the substrate of acid sphingomyelinase can not be degraded, but accumulates within the lysosomes of the affected organs of NPD patients. Depending on residual acid sphingomyelinase (aSMase) activity, individuals with an inborn defect in the aSMase gene, develop Type A or B NPD. The infantile Type A (aSMase activity less than 3% of normal) is the more severe form and is characterized by neuronal involvement and death by the age of 2 or 3 years. Type B NPD (less than 6% of normal aSMase activity) is the juvenile non-neuronopathic form of the disease and patients may survive into adulthood. Enzymatic activity above a threshold level of about 10% usually results in a complete or at least sufficient sphingomyelin turnover without any pathological phenotype. Thus, only a small increase in residual enzyme activity could have a significant impact on disease development and on life quality of NPD patients. Especially the milder forms of lysosomal storage disorders like NPD Type B are likely to be protein misfolding diseases, because alterations within the active site of an enzyme normally results in a complete loss of activity. A new, but very promising approach to treat lysosomal storage disorders is the use of small molecule substrate analogues or competitive inhibitors as chemical chaperones. The benefit of chemical chaperones is to protect variant enzymes from being degraded by the proteasome and to facilitate their transport to the lysosomes, thereby rescuing enzymatic activity. The rationale of this approach is the fact that variant lysosomal enzymes produced as a consequence of an inborn genetic mutation in the aSMase gene might be active in the acid environment of the lysosomes if only they could get there. Chemical chaperone mediated protection of variant enzymes occurs probably due to stabilisation of the native state fold of an otherwise misfolded enzyme by binding to its active site. Because of the very different chemical environments in the ER and in the lysosomes, some variant enzymes, which do not fold properly in the ER might retain partial or even full catalytic activity within the acidic chemical environment of the lysosomes. After an enzyme-inhibitor complex has reached the lysosome, the inhibitor is replaced by the accumulated substrate competing with the inhibitor to bind to the active site of the enzyme. Thus, paradoxically, an inhibitor of an enzyme in vitro can act as an enzyme activator in vivo.

The concept of chemical chaperones for the treatment of lysosomal storage disorders has first been described by Fan et al. for Fabry disease. Application of 1-deoxy-galactonojirimycin (DGJ) an inhibitor of α-galactosidase A (α-Gal A) effectively enhanced activity of this enzyme in Fabry lymphoblasts and in transgenic mice overexpressing a human Fabry variant of α-Gal A. Furthermore, injection of galactose, a much weaker inhibitor of α-Gal A to a cardiac Fabry patient resulted in considerable regression of pathology. It revealed that DGJ is able to stimulate α-Gal A seven- to eight-fold in cells when used at sub-inhibitory intracellular concentrations.

In fact, it was demonstrated that a potent inhibitor provides an effective chaperone, whereas less potent inhibitors require higher concentrations to achieve the same effect. This notion is most important, since potent inhibitors are expected to have therapeutic effects at lower concentrations that interact more specifically with the enzyme. By contrast, higher concentrations of moderately potent inhibitors are more likely to cross-react with other proteins.

Besides DGJ, which is in pre-clinical development, the concept has proven so far in cell culture with other substances for two further lysosomal storage disorders. Up to now, the exact mechanism by which chemical chaperones exhibit their function still remains to be elucidated. Recently, it has been found out that variant glucocerebrosidase characterized by destabilization of domains other than the catalytic domain is not amenable to stabilization by active site directed chemical chaperones. It is very likely that this observation reflects a general principle for chemical chaperones. Probably, stabilization of non active site domains may only be achieved by specifically designed molecules.

The potential for chemical chaperones to treat Niemann-Pick Disease has recently been outlined in WO 2005/051331.

Heat shock protein 70 (Hsp70) promotes the survival of cells, e.g. cancer cells, by stabilizing lysosomes, a hallmark of stress-induced cell death. (J. Nylandsted, M. Gyrd-Hansen, A. Danielewicz et al., J Exp Med (2004) 200 (4), 425). In cancer, a portion of Hsp70 translocates to the lysosomal compartment. It could be shown that Hsp70 stabilizes lysosomes by enhancing the activity of lysosomal acid sphingomyelinase. The pharmacological and genetic inhibition of aSMase effectively reverts the Hsp70-mediated stabilization of lysosomes (T. Kirkegaard, A. G. Roth, N. H. T. Petersen, A. K. Mahalka, O. D. Olsen, I. Moilanen, A. Zylicz, J. Knudsen, K. Sandhoff, C. Arenz, P. K. J. Kinnunen, J. Nylandsted, M. Jäättelä (2005) Hsp70 stabilizes lysosomes and reverts Niemann-Pick disease-associated lysosomal pathology, Nature 463, 549-554). Thus, inhibitors of aSMase sensitize cancer cells and tumours to chemo- or radiotherapy and therefore can be used in treatment, diagnosis and/or prophylaxis of cancer.

Thus, compounds of Formula I or preferred compounds thereof can be used in treatment, diagnosis and/or prophylaxis of infectious diseases, bacterial infections, infection with Neisseria gonnorhoeae, infections associated with cystic fibrosis, bacterial infections associated with cystic fibrosis, lung diseases, acute lung injury, acute respiratory distress syndrome, lung oedema, pulmonary emphysema, cystic fibrosis, Morbus Wilson, atherosclerosis, coronary heart disease, cardiovascular diseases, diabetes type II, depression, Alzheimer disease and/or Niemann-Pick disease and cancer. Preferably compounds of Formula I or preferred compounds thereof can be used in treatment, diagnosis and/or prophylaxis of infection with Neisseria gonnorhoeae, lung diseases, acute lung injury, acute respiratory distress syndrome, lung oedema, pulmonary emphysema, cystic fibrosis, Morbus Wilson, atherosclerosis, coronary heart disease, cardiovascular diseases, diabetes type II, depression, Alzheimer disease and/or Niemann-Pick disease. More preferably, compounds of Formula I or preferred compounds thereof can be used in treatment, diagnosis and/or prophylaxis of lung diseases, acute lung injury, acute respiratory distress syndrome, lung oedema, pulmonary emphysema and/or cystic fibrosis. Even more preferably, compounds of Formula I or preferred compounds thereof can be used in treatment, diagnosis and/or prophylaxis of acute lung injury and/or lung oedema.

Particular preferred compounds of Formula I can be used as medicament, wherein the particular preferred compound is
H₃C(CH₂)₄C(PO₃H₂)₂H
H₃C(CH₂)₅C(PO₃H₂)₂H
H₃C(CH₂)₆C(PO₃H₂)₂H
H₃C(CH₂)₇C(PO₃H₂)₂H
H₃C(CH₂)₈C(PO₃H₂)₂H
H₃C(CH₂)₉C(PO₃H₂)₂H
H₃C(CH₂)₁₀C(PO₃H₂)₂H
H₃C(CH₂)₁₁C(PO₃H₂)₂H
H₃C(CH₂)₆C(PO₃H₂)₂OH
H₃C(CH₂)₁₁C(PO₃H₂)₂OH
H₃C(CH₂)₆C(PO₃H₂)₂NH₂
H₃C(CH₂)₇C(PO₃H₂)₂NH₂
H₃C(CH₂)₈C(PO₃H₂)₂NH₂
H₃C(CH₂)₉C(PO₃H₂)₂NH₂
H₃C(CH₂)₁₀C(PO₃H₂)₂NH₂
H₃C(CH₂)₁₁C(PO₃H₂)₂NH₂
H₃C(CH₂)₄C(PO₃H₂)₂N(CH₃)₂
H₃C(CH₂)₅C(PO₃H₂)₂N(CH₃)₂
H₃C(CH₂)₆C(PO₃H₂)₂N(CH₃)₂
H₃C(CH₂)₇C(PO₃H₂)₂N(CH₃)₂
H₃C(CH₂)₈C(PO₃H₂)₂N(CH₃)₂
H₃C(CH₂)₉C(PO₃H₂)₂N(CH₃)₂
H₃C(CH₂)₁₀C(PO₃H₂)₂N(CH₃)₂
H₃C(CH₂)₁₁C(PO₃H₂)₂N(CH₃)₂
H₃C(CH₂)₄C(PO₃H₂)(PO₄H₂)H
H₃C(CH₂)₅C(PO₃H₂)(PO₄H₂)H
H₃C(CH₂)₆C(PO₃H₂)(PO₄H₂)H
H₃C(CH₂)₇C(PO₃H₂)(PO₄H₂)H
H₃C(CH₂)₈C(PO₃H₂)(PO₄H₂)H
H₃C(CH₂)₉C(PO₃H₂)(PO₄H₂)H
H₃C(CH₂)₁₀C(PO₃H₂)(PO₄H₂)H
H₃C(CH₂)₁₁C(PO₃H₂)(PO₄H₂)H
H₃C(CH₂)₄C(PO₃H₂)(PO₄H₂)OH
H₃C(CH₂)₅C(PO₃H₂)(PO₄H₂)OH
H₃C(CH₂)₆C(PO₃H₂)(PO₄H₂)OH
H₃C(CH₂)₇C(PO₃H₂)(PO₄H₂)OH
H₃C(CH₂)₈C(PO₃H₂)(PO₄H₂)OH
H₃C(CH₂)₉C(PO₃H₂)(PO₄H₂)OH
H₃C(CH₂)₁₀C(PO₃H₂)(PO₄H₂)OH
H₃C(CH₂)₁₁C(PO₃H₂)(PO₄H₂)OH
H₃C(CH₂)₄C(PO₃H₂)(PO₄H₂)NH₂
H₃C(CH₂)₅C(PO₃H₂)(PO₄H₂)NH₂
H₃C(CH₂)₆C(PO₃H₂)(PO₄H₂) NH₂
H₃C(CH₂)₇C(PO₃H₂)(PO₄H₂)NH₂
H₃C(CH₂)₈C(PO₃H₂)(PO₄H₂)NH₂
H₃C(CH₂)₉C(PO₃H₂)(PO₄H₂)NH₂
H₃C(CH₂)₁₀C(PO₃H₂)(PO₄H₂)NH₂
H₃C(CH₂)₁₁C(PO₃H₂)(P₄H₂)NH₂
H₃C(CH₂)₄C(PO₃H₂)(PO₄H₂)N(CH₃)₂
H₃C(CH₂)₅C(PO₃H₂)(PO₄H₂)N(CH₃)₂
H₃C(CH₂)₆C(PO₃H₂)(PO₄H₂)N(CH₃)₂
H₃C(CH₂)₇C(PO₃H₂)(PO₄H₂)N(CH₃)₂
H₃C(CH₂)₈C(PO₃H₂)(PO₄H₂)N(CH₃)₂
H₃C(CH₂)₉C(PO₃H₂)(PO₄H₂)N(CH₃)₂
H₃C(CH₂)₁₀C(PO₃H₂)(PO₄H₂)N(CH₃)₂; and/or
H₃C(CH₂)₁₁C(PO₃H₂)(PO₄H₂)N(CH₃)₂.

A compound of Formula I or a preferred compound thereof can be used for the preparation of a medicament for inhibition of acid sphingomyelinase enzyme activity.

A compound of Formula I or a preferred compound thereof can be used for the preparation of a medicament for treatment, diagnosis and/or prophylaxis of the diseases mentioned above.

The present invention also refers to a method of treatment, diagnosis or prophylaxis of a disease associated with aSMase activity and/or a disease mentioned above, comprising the administration of an effective amount of a compound of Formula I or a preferred compound thereof. An effective amount is an amount that yields to a measurable result with regard to treatment, diagnosis or prophylaxis of a disease associated with aSMase activity.

The present invention is also directed to a preferred compound of Formula I, wherein the preferred compound is
H₃C(CH₂)₄C(PO₃H₂)₂H
H₃C(CH₂)₆C(PO₃H₂)₂H
H₃C(CH₂)₇C(PO₃H₂)₂H
H₃C(CH₂)₈C(PO₃H₂)₂H
H₃C(CH₂)₆C(PO₃H₂)₂H
H₃C(CH₂)₁₀C(PO₃H₂)₂H
H₃C(CH₂)₁₁C(PO₃H₂)₂H
H₃C(CH₂)₉C(PO₃H₂)₂NH₂
H₃C(CH₂)₁₁C(PO₃H₂)₂NH₂
H₃C(CH₂)₆C(PO₃H₂)₂N(CH₃)₂
H₃C(CH₂)₇C(PO₃H₂)₂N(CH₃)₂
H₃C(CH₂)₈C(PO₃H₂)₂N(CH₃)₂
H₃C(CH₂)₉C(PO₃H₂)₂N(CH₃)₂
H₃C(CH₂)₁₀C(PO₃H₂)₂N(CH₃)₂;
H₃C(CH₂)₁₁C(PO₃H₂)₂N(CH₃)₂;
H₃C(CH₂)₄C(PO₃H₂)(PO₄H₂)H
H₃C(CH₂)₅C(PO₃H₂)(PO₄H₂)H
H₃C(CH₂)₆C(PO₃H₂)(PO₄H₂)H
H₃C(CH₂)₇C(PO₃H₂)(PO₄H₂)H
H₃C(CH₂)₈C(PO₃H₂)(PO₄H₂)H
H₃C(CH₂)₉C(PO₃H₂)(PO₄H₂)H
H₃C(CH₂)₁₀C(PO₃H₂)(PO₄H₂)H
H₃C(CH₂)₁₁C(PO₃H₂)(PO₄H₂)H
H₃C(CH₂)₄C(PO₃H₂)(PO₄H₂)OH
H₃C(CH₂)₅C(PO₃H₂)(PO₄H₂)OH
H₃C(CH₂)₆C(PO₃H₂)(PO₄H₂)OH
H₃C(CH₂)₇C(PO₃H₂)(PO₄H₂)OH
H₃C(CH₂)₈C(PO₃H₂)(PO₄H₂)OH
H₃C(CH₂)₉C(PO₃H₂)(PO₄H₂)OH
H₃C(CH₂)₁₀C(PO₃H₂)(PO₄H₂)OH
H₃C(CH₂)₁₁C(PO₃H₂)(PO₄H₂)OH
H₃C(CH₂)₄C(PO₃H₂)(PO₄H₂)NH₂
H₃C(CH₂)₅C(PO₃H₂)(PO₄H₂)NH₂
H₃C(CH₂)₆C(PO₃H₂)(PO₄H₂)NH₂
H₃C(CH₂)₇C(PO₃H₂)(PO₄H₂)NH₂
H₃C(CH₂)₈C(PO₃H₂)(PO₄H₂)NH₂
H₃C(CH₂)₉C(PO₃H₂)(PO₄H₂)NH₂
H₃C(CH₂)₁₀C(PO₃H₂)(PO₄H₂)NH₂
H₃C(CH₂)₁₁C(PO₃H₂)(PO₄H₂)NH₂
H₃C(CH₂)₄C(PO₃H₂)(PO₄H₂)N(CH₃)₂
H₃C(CH₂)₅C(PO₃H₂)(PO₄H₂)N(CH₃)₂
H₃C(CH₂)₆C(PO₃H₂)(PO₄H₂)N(CH₃)₂
H₃C(CH₂)₇C(PO₃H₂)(PO₄H₂)N(CH₃)₂
H₃C(CH₂)₈C(PO₃H₂)(PO₄H₂)N(CH₃)₂
H₃C(CH₂)₉C(PO₃H₂)(PO₄H₂)N(CH₃)₂
H₃C(CH₂)₁₀C(PO₃H₂)(PO₄H₂)N(CH₃)₂; and/or
H₃C(CH₂)₁₁C(PO₃H₂)(PO₄H₂)N(CH₃)₂.

### FIGURES

FIG. 1 shows that the aSMase inhibitor 7c (0.1 μM) inhibits dexamethasone (Dex)-induced apoptosis in HepG2 cells. The data refer to absorbance in a DNA-fragmentation ELISA.

FIG. 2 shows that the aSMase inhibitor 7c reduces PAF-induced pulmonary edema in isolated, ventilated and perfused rat lungs (IPL). Weight gain was measured 10 min after PAF-donation (5 nM).

FIG. 3 shows that the aSMase inhibitor 7c does not significantly inhibit PP1 activity when used in concentrations up to 2 μM.

### EXAMPLES

### Example 1

### Bisphosphonates and Mixed Phosphonate/Phosphate Compounds of Formula I and Formula II are Potent and Selective Inhibitors of aSMase

A collection of (bis)phosphonates that contained some compounds structurally-related to PtdIns-3,5P₂ were tested for their ability to inhibit aSMase. When theses substances were initially tested at 20 μM concentrations, it was found surprisingly that these compounds inhibited aSMase very potently (Tables 1 & 2). Among these substances, the geminal α-aminobisphosphonate 7b turned out to be about one order of magnitude more potent than PtdIns-3,5P₂. Furthermore, 7b in comparison with 7a, only consists of two additional methylene units, which leads to a dramatic increase in inhibitory potency.

TABLE 1
Inhibition of aSMase by the initial phosphonate collection

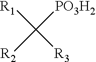

| Com- | | | | Inhibition [%] |
|---|---|---|---|---|
| pound | R₁ | R₂ | R₃ | at 20 μM^{[a]} |
| 1 | 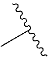 | H | H | 16 |
| 2 | 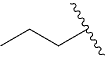 | H | H | 2 |
| 3 | 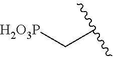 | NH₂ | H | 47 |
| 4 | 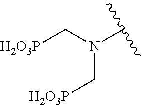 | H | H | 0 |
| 5 | 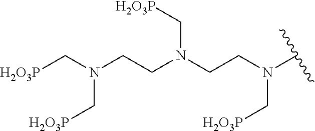 | H | H | −5 |

TABLE 2
Inhibition of aSMase by the initial bisphosphonate collection

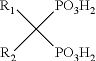

| | | | Inhibition [%] |
|---|---|---|---|
| Compound | R₁ | R₂ | at 20 μM |
| 6 | 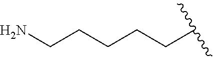 | OH | 54 |
| 7a | 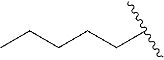 | NH₂ | 92 |
| 7b | 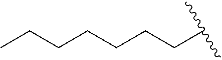 | NH₂ | 93 |
| 8 | 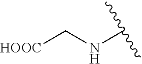 | CH₃ | 62 |
| 9 | 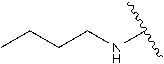 | H | 76 |
| 10 | 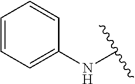 | H | 8 |
| 11 | 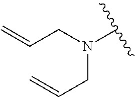 | H | 32 |
| 12 | 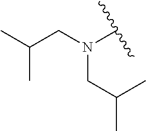 | H | 2 |
| 13 | 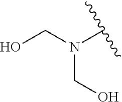 | H | 36 |
| 14 | 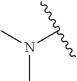 | H | 24 |

In order to gain a deeper insight into the structure activity relationship, a battery of 15 additional bisphosphonates were synthesized harbouring different functional groups at the α-carbon and displaying lipid-tails of different length, respectively (Scheme 1). Most of the syntheses were one- or two-step procedures yielding up to gram-amounts of the inhibitors according to well-established protocols (a) D. A. Nicholson, H. Vaughn, J. Org. Chem. 1971, 36, 3843. b) L. M. Nguyen, E. Niesor, C. L. Bentzen, J Med Chem 1987, 30, 1426. c) G. R. Kieczykowski, R. B. Jobson, D. G. Melillo, D. F. Reinhold, V. J. Grenda, I. Shinkai, J. Org. Chem. 1995, 60, 8310. d) D. V. Griffiths, J. M. Hughes, J. W. Brown, J. C. Caesar, S. P. Swetnam, S. A. Cumming, J. D. Kelly, Tetrahedron 1997, 53, 17815. e) S. H. Szajnman, E. L. Ravaschino, R. Docampo, J. B. Rodriguez, Bioorg Med Chem Lett 2005, 15, 4685.). On the basis of this new collection of compounds, it could be shown that inhibition correlates with the length of the lipid tail (this correlation is true as long as the substances are well-soluble) and that a functional group with free electron pairs at the α-carbon (—NH₂ more strongly than —OH) leads to an additionally increased inhibition of acid sphingomyelinase, when compared to the H-bisphosphonates 15a-d. Moreover, zoledronic acid 20, a widely-used drug against osteoporosis showed a marked inhibition of aSMase with an IC₅₀ value of approximately 5 μM (Table 3).

TABLE 3
Inhibition of aSMase by the synthesized bisphosphonates^{[a]}

| Entry | IC₅₀ [μM] |
|---|---|
| 7a | 4.66 ± 1.07 |
| 7b | 0.04 ± 0.01 |
| 7c | 0.02 ± 0.00 |
| 7d | 0.29 ± 0.09 |
| 15a | 0.35 ± 0.08 |
| 15b | 0.31 ± 0.12 |
| 15c | 0.30 ± 0.05 |
| 15d | 0.17 ± 0.04 |
| 16 | >100 |
| 17 | 0.35 ± 0.06 |
| 18a | 0.16 ± 0.04 |
| 18b | 0.08 ± 0.01 |
| 18c | 0.07 ± 0.01 |
| 18d | 6.80 ± 2.40 |
| 18e | 1.95 ± 0.22 |
| 19a | 9.50 ± 4.00 |
| 19b | 0.18 ± 0.03 |
| 20 | 5.08 ± 0.74 |

^{[a]}The IC₅₀ value for inhibition of nSMase was >100 μM for all compounds.

Bisphosphonates are known to form bidentate complexes with Me²⁺-ions like Ca²⁺, Zn²⁺ and Mg²⁺. With an additional hydroxyl or amine group, even more stable tridentate complexes can be formed. In fact, α-amino substitution leads to more stable complexes than an α-hydroxyl substitution, suggesting that aSMase inhibition also correlates with the tendency of the compounds to form complexes with the Zn²⁺ residing in the reactive center of the aSMase. It is noteworthy that aSMase, both in its lysosomal and its secreted form, is a Zn²⁺-dependent enzyme. However, the lysosomal variant is not inhibited by EDTA and not stimulated by Zn²⁺, which can be explained by abundance of Zn²⁺ in the lysosomes, whereas the secreted variant is stimulated by Zn²⁺. In order to characterize the aSMase-inhibiting bisphosphonates with regard to their metal-binding properties, compound 7c was tested in presence of millimolar concentrations of Ca²⁺, Mg²⁺ or Zn²⁺, respectively. The inhibitory activity was not significantly diminished by the metal ions.

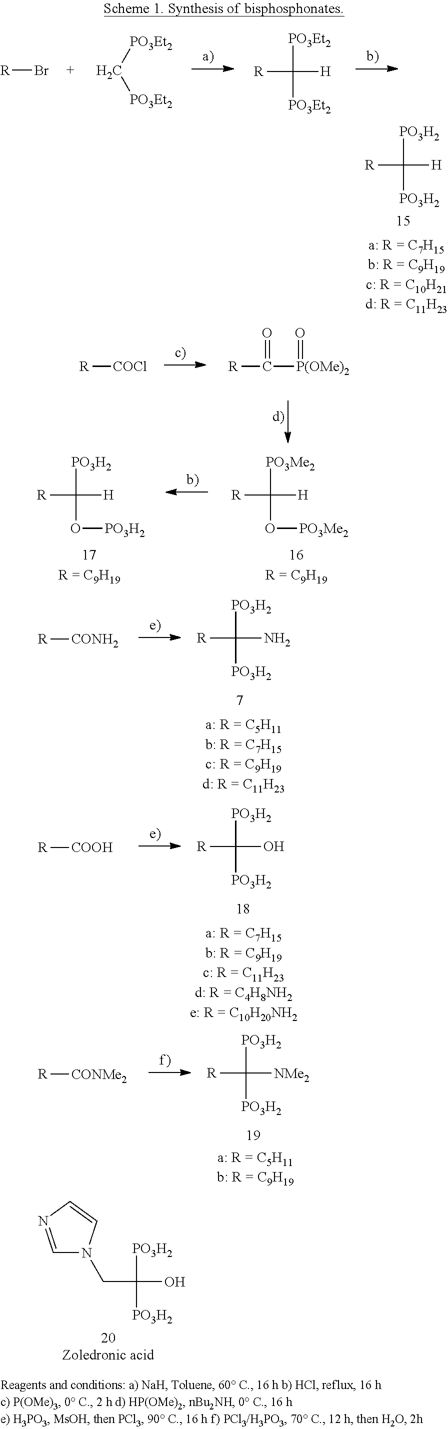

In addition, virtually all substances were tested for an inhibition of the Mg²⁺-dependent nSMase, without observing any substantial inhibition of this isoenzyme at concentrations up to 100 μM, clearly indicating that inhibition of aSMase is not only very potent, but is at the same time highly selective against nSMase.

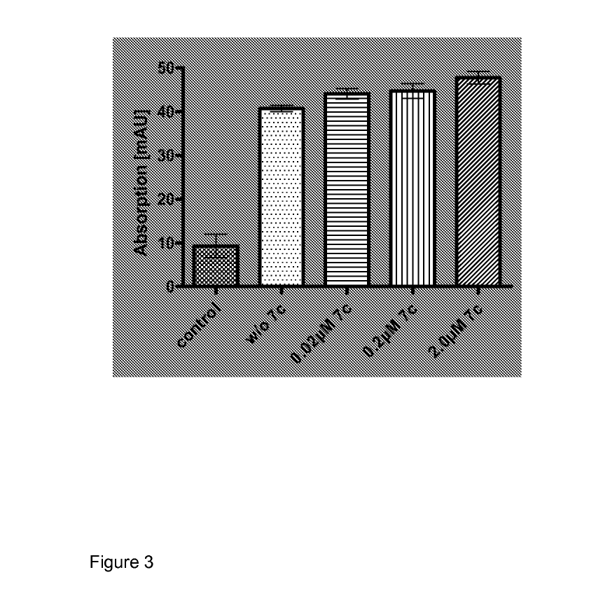

Moreover, the aSMase inhibitor 7c was tested for any inhibitory effect on the Ser/Thr phosphatase 1 (PP1), which—like the phosphodiesterase domain of aSMase—belongs to a family of dimetal-containing phosphoesterases. The PP1 enzyme was not inhibited by 7c, even at a concentration of 2 μM, which shows that this aSMase inhibitor is selective vs. PP1 (see FIG. 3).

To test for the influence of the negatively charged residues, the mixed phosphate/phosphonate compounds 16 and 17 were synthesized and tested. Whereas the mixed phosphate/phosphonate compound 17 is as active as its bisphosphonate analogue 15b, the methyl ester 16 is totally inactive towards aSMase, suggesting that aSMase inhibition is dependent on the metal complexing properties of the bisphosphonates.

### Example 2

### Inhibition of aSMase Activity can Efficiently Inhibit Apoptosis

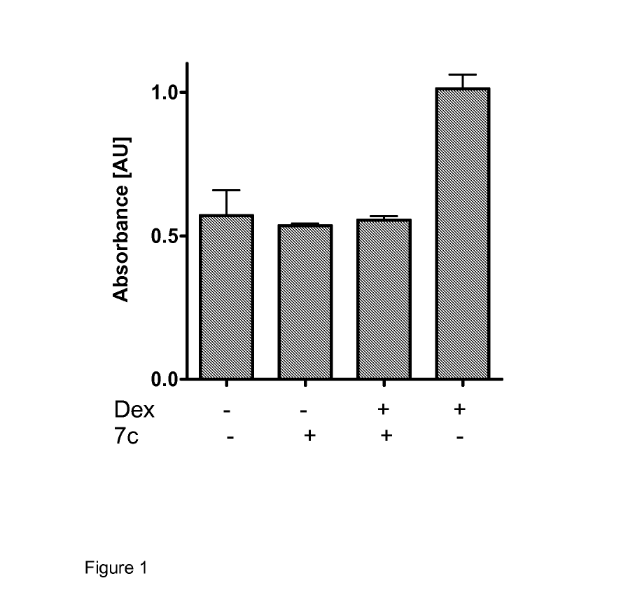

HepG2 liver cells were treated with dexamethasone (10⁻⁸M) in order to induce apoptosis, 0.1 μM of the aSMase inhibitor 7c efficiently inhibited apoptosis, as measured with a commercially-available DNA-fragmentation ELISA (FIG. 1).

### Example 3

### Inhibition of aSMase Activity can Inhibit Pulmonary Oedema

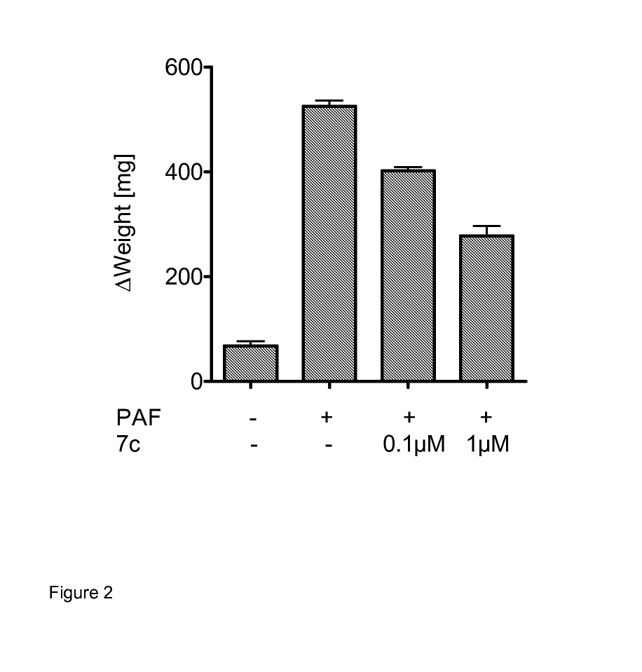

Encouraged by the high biological activity in cultured cells and because of the evident pharmacological interest in potent aSMase inhibitors for the treatment of lung diseases, it was examined whether inhibition of aSMase is also able to reduce PAF-induced pulmonary oedema, similar to the unspecific and indirect aSMase inhibitor imipramine (S. Uhlig, E. Gulbins, Am. J. Respir. Crit. Care Med. 2008, 178, 1100). Indeed, addition of 7c to the perfusate was concentration-dependently reduced oedema formation in isolated, ventilated and perfused rat lungs (IPL, shown in FIG. 2). Like imipramine (10 μM), the inhibitor 7c attenuated but not completely prevented oedema formation in this model.

The simple bisphosphonate 7c is the most potent aSMase inhibitor found so far. It is more than 5.000 fold selective against the Mg²⁺-dependent isoenzyme nSMase and selective against the dimetal-containing remote aSMase-homologue Ser/Thr protein phosphatase 1. The compound, which easily can be synthesized in gram-scale is also active in cell culture and efficiently protects HepG2 cells from dexamethasone-induced apoptosis.

### Experimental Procedures

Enzyme assays: Crude preparations containing aSMase or nSMase were made from stripped rat brains, as described before. The micellar nSMase assays using ¹⁴C-labeled sphingomyelin as a substrate were performed as described before (V. Wascholowski, A. Giannis, Angew. Chem., 2006, 118, 841; Angew Chem Int Ed 2006, 45, 827). The fluorescent aSMase assay was performed in a 384-well-plate using the HMU-PC (6-Hexadecanoylamino-4-methylumbelliferyl-phosphorylcholine) substrate. Reaction mixtures consisted of 13.3 μL HMU-PC, 13.3 μL reaction-buffer (100 mM NaOAc, pH 5.2, 0.2% (w/v) Na—TC, 0.02% (w/v), 0.2% (v/v) Triton X-100) and 13.3 μL enzyme preparation. Inhibitors were added in various concentrations and the reactions were incubated for 3 hours at 37° C. in a plate reader (FLUOstar OPTIMA, BMG labtech). The fluorescence of HMU (6-Hexadecanoylamino-4-methylumbelliferone) was measured (excitation 380 nm, emission 460 nm) in real time. Assays using the radio-labelled sphingomyelin gave the same results.

Compound libraries and syntheses: all described compounds were verified using ¹H-, ¹³C- and ³¹P-NMR and MS, respectively. The syntheses were accomplished as described before (a) D. A. Nicholson, H. Vaughn, J. Org. Chem. 1971, 36, 3843. b) L. M. Nguyen, E. Niesor, C. L. Bentzen, J Med Chem 1987, 30, 1426. c) G. R. Kieczykowski, R. B. Jobson, D. G. Melillo, D. F. Reinhold, V. J. Grenda, I. Shinkai, J. Org. Chem. 1995, 60, 8310. d) D. V. Griffiths, J. M. Hughes, J. W. Brown, J. C. Caesar, S. P. Swetnam, S. A. Cumming, J. D. Kelly, Tetrahedron 1997, 53, 17815. e) S. H. Szajnman, E. L. Ravaschino, R. Docampo, J. B. Rodriguez, Bioorg Med Chem Lett 2005, 15, 4685).

Apoptosis assay: First, the kinetics of DNA fragmentation after dexamethasone-donation was measured in the lysate and in the supernatant, respectively. Between 6 h and 8 h, there was a steep increase in absorbance in the probes from the supernatant, which is typical for apoptosis (data not shown). The apoptosis assay was performed according to the manufacturer's protocol (Roche cat. No. 11585045). Briefly, cells were harvested and suspended in culture medium (2×10⁵ cells/ml) containing BrdU labelling solution (10 μM final concentration) and plated in a 96-well cell culture dish at ˜1×10⁴ cells per well. After 16 h, cells were washed and new media was added. Then, cells were treated with 10⁻⁸ M of dexamethasone and 0.1 μM of 7c, respectively. After 7 hours of incubation, 100 μl of the supernatant was collected and added to a 96well plate containing immobilized anti-BrdU antibody. After incubation, removal of the supernatant and extensive washing, the secondary antibody and the TMB substrate were added and absorbance was measured at 370 nm (FLUOstar OPTIMA, BMG labtech). The experiment was performed in quintuplicate.

PAF-induced pulmonary edema: Female Wistar rats (weight 220 to 250 g) were kept on a standard laboratory chow and water ad libitum. Rat lungs were prepared, perfused and ventilated essentially as described (S. Uhlig, E. Gulbins, Am. J. Respir. Crit. Care Med. 2008, 178, 1100). Briefly, lungs were perfused through the pulmonary artery at a constant hydrostatic pressure (12 cm H₂O) with Krebs-Henseleit-buffer containing 2% albumin, 0.1% glucose and 0.3% HEPES. Edema formation was assessed by continuously measuring the weight gain of the lung. In this model, platelet-activating factor causes rapid edema formation that is in part dependent on acid sphingomyelinase. 7c was dissolved in buffer and added to the buffer reservoir 10 min prior to PAF (5 nMol) administration. Isolated perfused rat lungs were perfused for 30 min before 7c was added to the perfusate. 10 min later 5 nMol PAF was added as a bolus and weight gain was followed for 10 min. Data are shown as mean±SD from 4 independent experiments in each group. Statistics: 0.1 μM 7c: p<0.01 vs PAF alone; 1 μM 7c: p<0.01 vs. PAF alone and vs. 0.1 μM 7c/PAF (Tukey's Test).

PP1 assay: The protein phosphatase 1 (PP1, New England Biolabs P0754L) activity was assayed in a reaction mixture of 50 μL according to the manufacturer's conditions, but containing only 1% (500 μM) of the recommended amount of p-nitrophenylphosphate (PNPP, New England Biolabs P0757L). In a preceding experiment the K_{M} for PNPP was determined to be 3 mM (data not shown), which is in agreement with the manufacturer's statement (Km=0.5 to 10 mM for all phosphatases). Briefly, the substrate and various inhibitor concentrations were added to the reaction buffer containing 1 mM MnCl₂, 50 mM HEPES, 100 mM NaCl, 0.1 mM EGTA, 2 mM dithiothreitol, 0.025% Tween 20 at pH 7.5. The reaction was initiated by addition of PP1 (1.25 U). After 6 min the reaction was quenched by addition of 10 μl of 0.5M EDTA-solution (pH 8). The amount of the formed product, p-nitrophenol, was determined by measuring the absorbance at 405 nm (Nanodrop). The control was composed as described above, including 0.2 μM 7c but with heat-denatured enzyme. All measurements were done at least in triplicate.

### Procedures for the Synthesis of Previously Unknown Substances (16, 17 and 19 a/b:)

### Procedure for Preparation of 1-dimethylaminodecyl-1,1-bisphosphonicacid (19b)

N,N-Dimethyldecanamide (1.0 g, 5.02 mmol) was slowly added to an initially stirred mixture of phosphorus trichloride (1.0 ml, 11.4 mmol) and phosphorous acid (0.42 g, 5.12 mmol). The mixture was heated at 70° C. for 2 h. After cooling the excess phosphorous trichloride was decanted off and the residue hydrolyzed by the careful addition of plenty of water. This mixture was left to stir for at least 2 h, filtered, and the filtrate evaporated to dryness under reduced pressure. The precipitate was taken up in 20 ml of water and heated at 100° C. for 1 h, followed by filtration of the hot solution. The water was evaporated and the desired product was isolated as a colorless solid (1.73 g, quant). ¹H NMR (300 MHz, D₂O): δ=0.84 (t, J=6.72 Hz, 3H), 1.26 (m, 8H), 1.31 (d, J=3.94 Hz, 4H), 1.55 (dd, J=2.28, 4.34 Hz, 2H), 2.00 (m, 2H), 3.063 (s, 6H) ppm. ¹³C NMR (75 MHz, D₂O): δ=13.46, 22.16, 28.69, 28.75, 28.92, 29.14, 30.06, 31.34, 31.41, 41.80, 70.04 (t, J=108.65 Hz, 1C) ppm. ³¹P NMR (121 MHz, D₂O): δ=3.71 ppm. HRMS: m/z calcd. for C₁₂H₂₈NO₆P₂: 344.1397. found: 344.1389.

### Procedure for Preparation of 1-Dimethylaminodecyl-1,1-bisphosphonicacid (19a)

N,N-Dimethylhexanamide (1.5 g, 10.6 mmol) was slowly added to an initially stirred mixture of phosphorus trichloride (2.8 ml, 32.2 mmol) and phosphorous acid (1.15 g, 14.0 mmol). The mixture was heated at 70° C. for 2 h. After cooling the excess phosphorous trichloride was decanted off and the residue hydrolyzed by the careful addition of plenty of water. This mixture was left to stir for at least 2 h, filtered, and the filtrate evaporated to dryness under reduced pressure. The precipitate was taken up in 20 ml of water and heated at 100° C. for 1 h, followed by filtration of the hot solution. The water was evaporated and the desired product was isolated as a colorless solid (1.64 g, 54%). ¹H NMR (300 MHz, D₂O): δ=0.83 (t, J=6.26 Hz, 3H), 1.27 (tt, J=3.50, 7.24 Hz, 4H), 1.50 (qd, J=7.04, 6.98, 8.70 Hz, 2H), 1.98-2.04 (m, 2H), 3.03 (s, 6H) ppm. ¹³C NMR (75 MHz, D₂O): δ=13.22, 21.58, 23.00, 28.93, 31.54, 42.07, 69.18 ppm. ³¹P NMR (121 MHz, D₂O): δ=4.66 ppm. HRMS: m/z calcd. for C₈H₂₂NO₆P₂: 290.0917. found: 290.0904.

### Procedure for Preparation of Decyl-1-dimethylphosphate-1-dimethylphosphohonate (16)

Decanoylchloride (4 g, 21.0 mmol) was placed in a mechanically stirred reaction flask and cooled to 0° C. Trimethylphosphite (2.60 g, 21.0 mmol) was added drop wise with rapid stirring (gas evolution). After addition was complete the reaction mixture was allowed to warm up at room temperature. The reaction mixture was evaporated under reduced pressure. To the colourless oil was added dimethylphosphite (1.15 g, 10.5 mmol) and ether (50 ml), followed by an addition of di-n-butylamine (0.14 g, 1.05 mmol) and cooling to 0° C. The reaction mixture was allowed to warm up at room temperature and was stirring over night. Purification of the crude product was purified by silica gel chromatography (dichloromethane/methanol 20:1) and gave the desired product in 16% (1.33 g) yield. ¹H NMR (300 MHz, CDCl₃): δ=0.67-0.76 (m, 3H), 1.11 (s, 12H), 1.25-1.45 (m, 2H), 1.65-1.80 (m, 2H), 1.82-1.90 (m, 1H), 3.55-3.73 (m, 12H) ppm. ¹³C NMR (75 MHz, CDCl₃): δ=13.91, 22.48, 24.99, 25.14, 28.94, 29.10, 29.15, 29.29, 30.65, 31.70, 53.21, 53.29, 54.33, 54.41, 71.65, 71.74, 73.89, 73.99 ppm. ³¹P NMR (121 MHz, CDCl₃): δ=1.57. 1.73. 22.95, 23.11 ppm. HRMS: m/z calcd. for C₁₄H₃₃O₇P₂: 375.1696. found: 375.1685.

### Procedure for preparation of Decyl-1,1-phosphate phosphonate (17)

The Dimethylphosphonate dimethylphosphateesters of compound 16 (0.35 g, 0.94 mmol) was hydrolyzed by refluxing for 8 h with an excess of concentrated hydrochloride acid. The acid was evaporated and the desired product was isolated as colorless oil in 99% (0.29 g, 0.91 mmol) yield. ¹H NMR (300 MHz, O₂O): δ=0.57 (t, J=6.37 Hz, 3H), 0.90-1.10 (m, 12H), 1.22-1.43 (m, 2H), 1.48-1.64 (m, 2H), 4.14 (s, 1H) ppm. ¹³C NMR (75 MHz, D₂O): δ=13.56, 22.40, 25.14, 25.28, 29.24, 29.33, 29.63, 30.46, 31.81, 72.31, 72.39, 74.48, 74.57 ppm. ³¹P NMR (121 MHz, D₂O): δ=0.22, 20.43 ppm. HRMS: m/z calcd. for C₁₀H₂₅O₇P₂: 319.1070. found: 319.1070.

1-9. (canceled)

10. A method of treatment, diagnosis and/or prophylaxis of lung disease, acute lung injury, acute respiratory distress syndrome, lung oedema, pulmonary emphysema and/or cystic fibrosis, comprising the administration of an effective amount of a compound of Formula I to a patient in need thereof,
wherein
Formula I is: 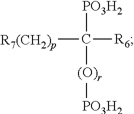
and wherein
p is an integer from 4 to 12;
r is 0 or 1;
R₆=H, OH, NH₂ or N(CH₃)₂; and
R₇=CH₃.

11. The method of claim 10, wherein the compound is used in treatment, diagnosis and/or prophylaxis of acute lung injury and/or lung oedema.

12. The method of claim 10, wherein p is an integer from 5 to 10.

13. The method of claim 10, wherein the compound is selected from the group consisting of:
H₃C(CH₂)₄C(PO₃H₂)₂H
H₃C(CH₂)₅C(PO₃H₂)₂H
H₃C(CH₂)₆C(PO₃H₂)₂H
H₃C(CH₂)₇C(PO₃H₂)₂H
H₃C(CH₂)₈C(PO₃H₂)₂H
H₃C(CH₂)₉C(PO₃H₂)₂H
H₃C(CH₂)₁₀C(PO₃H₂)₂H
H₃C(CH₂)₁₁C(PO₃H₂)₂H
H₃C(CH₂)₆C(PO₃H₂)₂OH
H₃C(CH₂)₁₁C(PO₃H₂)₂OH
H₃C(CH₂)₆C(PO₃H₂)₂NH₂
H₃C(CH₂)₇C(PO₃H₂)₂NH₂
H₃C(CH₂)₈C(PO₃H₂)₂NH₂
H₃C(CH₂)₉C(PO₃H₂)₂NH₂
H₃C(CH₂)₁₀C(PO₃H₂)₂NH₂
H₃C(CH₂)₁₁C(PO₃H₂)₂NH₂
H₃C(CH₂)₄C(PO₃H₂)₂N(CH₃)₂
H₃C(CH₂)₅C(PO₃H₂)₂N(CH₃)₂
H₃C(CH₂)₆C(PO₃H₂)₂N(CH₃)₂
H₃C(CH₂)₇C(PO₃H₂)₂N(CH₃)₂
H₃C(CH₂)₈C(PO₃H₂)₂N(CH₃)₂
H₃C(CH₂)₉C(PO₃H₂)₂N(CH₃)₂
H₃C(CH₂)₁₀C(PO₃H₂)₂N(CH₃)₂
H₃C(CH₂)₁₁C(PO₃H₂)₂N(CH₃)₂
H₃C(CH₂)₄C(PO₃H₂)(PO₄H₂)H
H₃C(CH₂)₅C(PO₃H₂)(PO₄H₂)H
H₃C(CH₂)₆C(PO₃H₂)(PO₄H₂)H
H₃C(CH₂)₇C(PO₃H₂)(PO₄H₂)H
H₃C(CH₂)₈C(PO₃H₂)(PO₄H₂)H
H₃C(CH₂)₉C(PO₃H₂)(PO₄H₂)H
H₃C(CH₂)₁₀C(PO₃H₂)(PO₄H₂)H
H₃C(CH₂)₁₁C(PO₃H₂)(PO₄H₂)H
H₃C(CH₂)₄C(PO₃H₂)(PO₄H₂)OH
H₃C(CH₂)₅C(PO₃H₂)(PO₄H₂)OH
H₃C(CH₂)₆C(PO₃H₂)(PO₄H₂)OH
H₃C(CH₂)₇C(PO₃H₂)(PO₄H₂)OH
H₃C(CH₂)₈C(PO₃H₂)(PO₄H₂)OH
H₃C(CH₂)₉C(PO₃H₂)(PO₄H₂)OH
H₃C(CH₂)₁₀C(PO₃H₂)(PO₄H₂)OH
H₃C(CH₂)₁₁C(PO₃H₂)(PO₄H₂)OH
H₃C(CH₂)₄C(PO₃H₂)(PO₄H₂)NH₂
H₃C(CH₂)₅C(PO₃H₂)(PO₄H₂)NH₂
H₃C(CH₂)₆C(PO₃H₂)(PO₄H₂)NH₂
H₃C(CH₂)₇C(PO₃H₂)(PO₄H₂)NH₂
H₃C(CH₂)₈C(PO₃H₂)(PO₄H₂)NH₂
H₃C(CH₂)₉C(PO₃H₂)(PO₄H₂)NH₂
H₃C(CH₂)₁₀C(PO₃H₂)(PO₄H₂)NH₂
H₃C(CH₂)₁₁C(PO₃H₂)(PO₄H₂)NH₂
H₃C(CH₂)₄C(PO₃H₂)(PO₄H₂)N(CH₃)₂
H₃C(CH₂)₅C(PO₃H₂)(PO₄H₂)N(CH₃)₂
H₃C(CH₂)₆C(PO₃H₂)(PO₄H₂)N(CH₃)₂
H₃C(CH₂)₇C(PO₃H₂)(PO₄H₂)N(CH₃)₂
H₃C(CH₂)₈C(PO₃H₂)(PO₄H₂)N(CH₃)₂
H₃C(CH₂)₉C(PO₃H₂)(PO₄H₂)N(CH₃)₂
H₃C(CH₂)₁₀C(PO₃H₂)(PO₄H₂)N(CH₃)₂; and
H₃C(CH₂)₁₁C(PO₃H₂)(PO₄H₂)N(CH₃)₂.

14. A method of inhibiting acid sphingomyelase in vitro, wherein a compound of Formula I is used,
with 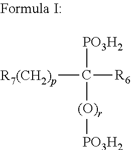
wherein
p is an integer from 4 to 12;
r is 0 or 1;
R₆=H, OH, NH₂ or N(CH₃)₂; and
R₇=CH₃, NH₂ or N(CH₃)₂; preferably R₇ is CH₃.

15. The method of claim 14, wherein the compound is
H₃C(CH₂)₄C(PO₃H₂)₂H
H₃C(CH₂)₅C(PO₃H₂)₂H
H₃C(CH₂)₆C(PO₃H₂)₂H
H₃C(CH₂)₇C(PO₃H₂)₂H
H₃C(CH₂)₈C(PO₃H₂)₂H
H₃C(CH₂)₉C(PO₃H₂)₂H
H₃C(CH₂)₁₀C(PO₃H₂)₂H
H₃C(CH₂)₁₁C(PO₃H₂)₂H
H₃C(CH₂)₄C(PO₃H₂)₂OH
H₃C(CH₂)₅C(PO₃H₂)₂OH
H₃C(CH₂)₆C(PO₃H₂)₂OH
H₃C(CH₂)₇C(PO₃H₂)₂OH
H₃C(CH₂)₈C(PO₃H₂)₂OH
H₃C(CH₂)₉C(PO₃H₂)₂OH
H₃C(CH₂)₁₀C(PO₃H₂)₂OH
H₃C(CH₂)₁₁C(PO₃H₂)₂OH
H₃C(CH₂)₄C(PO₃H₂)₂NH₂
H₃C(CH₂)₅C(PO₃H₂)₂NH₂
H₃C(CH₂)₆C(PO₃H₂)₂NH₂
H₃C(CH₂)₇C(PO₃H₂)₂NH₂
H₃C(CH₂)₈C(PO₃H₂)₂NH₂
H₃C(CH₂)₉C(PO₃H₂)₂NH₂
H₃C(CH₂)₁₀C(PO₃H₂)₂NH₂
H₃C(CH₂)₁₁C(PO₃H₂)₂NH₂
H₃C(CH₂)₄C(PO₃H₂)₂N(CH₃)₂
H₃C(CH₂)₅C(PO₃H₂)₂N(CH₃)₂
H₃C(CH₂)₆C(PO₃H₂)₂N(CH₃)₂
H₃C(CH₂)₇C(PO₃H₂)₂N(CH₃)₂
H₃C(CH₂)₈C(PO₃H₂)₂N(CH₃)₂
H₃C(CH₂)₉C(PO₃H₂)₂N(CH₃)₂
H₃C(CH₂)₁₀C(PO₃H₂)₂N(CH₃)₂
H₃C(CH₂)₁₁C(PO₃H₂)₂N(CH₃)₂
H₃C(CH₂)₄C(PO₃H₂)(PO₄H₂)H
H₃C(CH₂)₅C(PO₃H₂)(PO₄H₂)H
H₃C(CH₂)₆C(PO₃H₂)(PO₄H₂)H
H₃C(CH₂)₇C(PO₃H₂)(PO₄H₂)H
H₃C(CH₂)₈C(PO₃H₂)(PO₄H₂)H
H₃C(CH₂)₉C(PO₃H₂)(PO₄H₂)H
H₃C(CH₂)₁₀C(PO₃H₂)(PO₄H₂)H
H₃C(CH₂)₁₁C(PO₃H₂)(PO₄H₂)H
H₃C(CH₂)₄C(PO₃H₂)(PO₄H₂)OH
H₃C(CH₂)₅C(PO₃H₂)(PO₄H₂)OH
H₃C(CH₂)₆C(PO₃H₂)(PO₄H₂)OH
H₃C(CH₂)₇C(PO₃H₂)(PO₄H₂)OH
H₃C(CH₂)₈C(PO₃H₂)(PO₄H₂)OH
H₃C(CH₂)₉C(PO₃H₂)(PO₄H₂)OH
H₃C(CH₂)₁₀C(PO₃H₂)(PO₄H₂)OH
H₃C(CH₂)₁₁C(PO₃H₂)(PO₄H₂)OH
H₃C(CH₂)₄C(PO₃H₂)(PO₄H₂)NH₂
H₃C(CH₂)₅C(PO₃H₂)(PO₄H₂)NH₂
H₃C(CH₂)₆C(PO₃H₂)(PO₄H₂)NH₂
H₃C(CH₂)₇C(PO₃H₂)(PO₄H₂)NH₂
H₃C(CH₂)₈C(PO₃H₂)(PO₄H₂)NH₂
H₃C(CH₂)₉C(PO₃H₂)(PO₄H₂)NH₂
H₃C(CH₂)₁₀C(PO₃H₂)(PO₄H₂)NH₂
H₃C(CH₂)₁₁C(PO₃H₂)(PO₄H₂)NH₂
H₃C(CH₂)₄C(PO₃H₂)(PO₄H₂)N(CH₃)₂
H₃C(CH₂)₅C(PO₃H₂)(PO₄H₂)N(CH₃)₂
H₃C(CH₂)₆C(PO₃H₂)(PO₄H₂)N(CH₃)₂
H₃C(CH₂)₇C(PO₃H₂)(PO₄H₂)N(CH₃)₂
H₃C(CH₂)₈C(PO₃H₂)(PO₄H₂)N(CH₃)₂
H₃C(CH₂)₉C(PO₃H₂)(PO₄H₂)N(CH₃)₂
H₃C(CH₂)₁₀C(PO₃H₂)(PO₄H₂)N(CH₃)₂
H₃C(CH₂)₁₁C(PO₃H₂)(PO₄H₂)N(CH₃)₂
NH₂(CH₂)₄C(PO₃H₂)₂OH
NH₂(CH₂)₅C(PO₃H₂)₂OH
NH₂(CH₂)₆C(PO₃H₂)₂OH
NH₂(CH₂)₇C(PO₃H₂)₂OH
NH₂(CH₂)₈C(PO₃H₂)₂OH
NH₂(CH₂)₉C(PO₃H₂)₂OH
NH₂(CH₂)₁₀C(PO₃H₂)₂OH
NH₂(CH₂)₁₁C(PO₃H₂)₂OH
N(CH₃)₂(CH₂)₄C(PO₃H₂)₂OH
N(CH₃)₂(CH₂)₅C(PO₃H₂)₂OH
N(CH₃)₂(CH₂)₆C(PO₃H₂)₂OH
N(CH₃)₂(CH₂)₇C(PO₃H₂)₂OH
N(CH₃)₂(CH₂)₈C(PO₃H₂)₂OH
N(CH₃)₂(CH₂)₉C(PO₃H₂)₂OH
N(CH₃)₂(CH₂)₁₀C(PO₃H₂)₂OH and/or
N(CH₃)₂(CH₂)₁₁C(PO₃H₂)₂OH.
